(19) United States
(12) Patent Application Publication
(10) Pub. No.: US 2009/0036977 A1
(43) Pub. Date: Feb. 5, 2009

(54) DRUG-RELEASING STENT HAVING EXTENSION(S) FOR TREATING LONG LESIONS

(75) Inventors: Jay Rassat, Buffalo, MN (US); Derek Sutermeister, Plymouth, MN (US); Scott Petersen, Brooklyn Park, MN (US)

Correspondence Address: JONES DAY, 222 EAST 41ST ST, NEW YORK, NY, 10017, US

(73) Assignee: Boston Scientific Scimed, Inc., Maple grove, MN (US)

(21) Appl. No.: 12/103,750

(22) Filed: Apr. 16, 2008

Related U.S. Application Data
(60) Provisional application No. 60/912,224, filed on Apr. 17, 2007.

(51) Int. Cl. A61F 2/06

(52) U.S. Cl. 623/1.42; 623/1.15; 623/1.44

(57) ABSTRACT

This invention generally relates to a system for treating body lumens, comprising stents, such as intravascular stents. More particularly, the invention is directed to stents having extensions attached on the ends thereof. The invention is also directed to methods for making and using such stents.

### CROSS-REFERENCE TO RELATED APPLICATIONS

This application claims the benefit of U.S. Provisional Application No. 60/912,224, filed on Apr. 17, 2007, the entire contents of which are incorporated herein by reference.

### FIELD OF THE INVENTION

This invention generally relates to a system for treating body lumens, comprising stents, such as intravascular stents. More particularly, the invention is directed to stents having extensions attached to the ends thereof. The invention is also directed to methods for making and using such stents.

### BACKGROUND OF THE INVENTION

Stents are used to treat a variety of medical conditions. In blood vessels, they have been used to treat, e.g., stenoses and aneurysms. They have also been used to treat or correct conditions in body lumens other than blood vessels, such as the ureter, duodenum, and bile duct. Furthermore, stents have been used for the localized delivery of therapeutic agents to a body lumen. For example stents that incorporate or are coated with a therapeutic agent have been used for treating restenosis.

In certain patients the area of the lumen that is to be treated with the stent is longer or more extensive than the length of the stent. For instance, in the case of a blood vessel, the lesion on the blood vessel that requires treatment may extend beyond the length of a vascular stent that is typically used to treat such lesions. In such situations, several approaches have been employed. One approach is to use a longer stent. However, a longer stent can be difficult to deliver and deploy. Another approach is to use a series of two or more shorter stents that are overlapped or laid adjacent to one another to provide the required length. However, overlapping stents can cause problems such as lumen occlusion, re-occlusion, or restenosis.

Therefore, there is a need for a stent system and method for treating a lumen, having an extensive area that requires treatment, without the disadvantages of previous devices and methods for treating such lumens.

### SUMMARY OF THE INVENTION

The present invention seeks to address these objectives by providing, in one embodiment, a system for treating a lumen which comprises a first stent. This first stent has a surface, a first end and a second end. A coating composition comprising a first polymer and a first therapeutic agent is disposed on the surface of the first stent. There is also a first tubular extension attached to the second end of the first stent. This first extension comprises a second polymer and a second therapeutic agent. The system also includes a second stent having a surface, a first end and a second end. The first end of the second stent forms an overlap with the first extension. The system can include additional stent or addition extensions.

In another embodiment, the system for treating a blood vessel comprises a first intravascular metal stent having a surface, a first end, and a second end. A coating composition comprising a first polymer and an agent for inhibiting the proliferation of smooth muscle cells is disposed on the surface of the first stent. A first tubular extension is attached to the second end of the first stent. This first extension comprises a second polymer and the agent for inhibiting the proliferation of smooth muscle cells. In addition, the system includes a second intravascular metal stent having a surface upon which the coating composition is disposed. The second stent also has a first end and a second end, wherein the first end of the second stent forms an overlap with less than the entire first extension.

In yet another embodiment, the system for treating a blood vessel comprises a first intravascular metal stent having an abluminal surface, a first end, a second end, and a coating composition comprising a first biostable polymer and an anti-restenosis agent disposed on the abluminal surface. There is a first tubular extension attached to the second end of the first stent. The first extension comprises a second biostable polymer and the anti-restenosis agent. Additionally, the system includes a second intravascular metal stent having an abluminal surface, a first end and a second end. The first end of the second stent forms an overlap with less than the entire first extension.

Furthermore, the present invention is directed to a system for treating a bifurcated lumen comprising a bifurcated stent. The bifurcated stent comprises a surface, a first tubular portion, a second tubular portion, and a third tubular portion having an end. A tubular extension is attached to the end of the third tubular portion. The system also includes a non-bifurcated stent comprising a surface, a first end and a second end. The first end of the non-bifurcated stent forms an overlap the extension attached to the end of the third portion of the bifurcated stent.

In another embodiment, the system for treating a bifurcated blood vessel comprises a bifurcated intravascular metal stent comprising a surface, a first tubular portion, a second tubular portion, and a third tubular portion having an end. A tubular extension, which comprises a first polymer, is attached to the end of the third tubular portion. The system also includes a non-bifurcated intravascular metal stent comprising a surface, a first end and a second end. The first end of the non-bifurcated stent forms an overlap with less than the entire extension. Also, a graft comprising the first polymer is disposed within the bifurcated stent.

### BRIEF DESCRIPTION OF THE DRAWINGS

Preferred features of the present invention are disclosed in the accompanying drawings, wherein similar reference characters denote similar elements throughout the several views, and wherein:

FIGS. 1A and 1B show an exemplary stent system comprising a stent with an extension and a second stent.

FIGS. 2A and 2B show alternative ways in which the extension and the end of a stent overlap at A-A of FIG. 1B.

FIGS. 3A and 3B show another exemplary stent system comprising a first stent with an extension, a second stent with an extension, and a third stent.

FIGS. 4A and 4B show another exemplary stent system comprising a stent with two extensions and two other stents.

FIGS. 5A and 5B show another exemplary stent system comprising a stent with a tapered extension and a second stent.

FIGS. 6A and 6B show another exemplary stent system having a stent with a tapered extension and a second stent.

FIG. 7 shows a stent system comprising stents and extensions implanted in a curved and tapered blood vessel.

FIG. 8 shows a stent system comprising a bifurcated stent with an extension and a second non-bifurcated stent.

FIG. 9 shows the stent system of FIG. 8 implanted in a bifurcated vessel.

FIGS. 10A-10C show the delivery of a stent system to a lesion in a lumen.

### DETAILED DESCRIPTION OF THE INVENTION

Illustrative embodiments will now be described in detail.

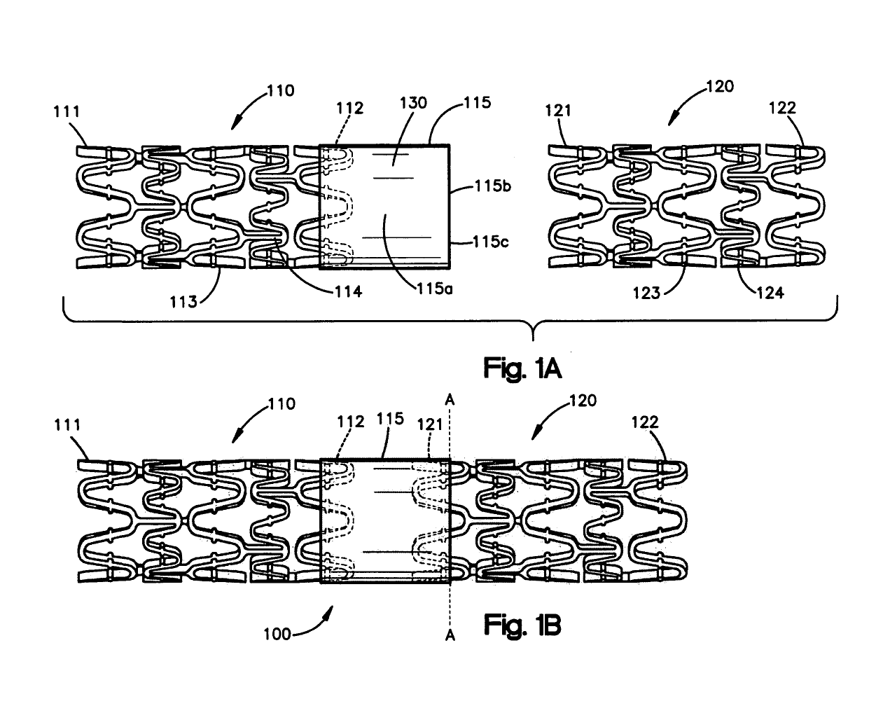

In one embodiment, which is shown in FIGS. 1A and 1B, a system 100 for treating a lumen comprises a first stent 110 with an extension 115 and a second stent 120.

FIG. 1A shows the system when the extension 115 and second stent 120 are not connected or in contact with each other. FIG. 1B shows the system when the extension 115 and the second stent 120 form an overlap. In this system, the first stent 110 has a surface 113, a first end 111 and a second end 112. A first extension 115 having a surface 115a is attached or connected to the second end 112 of the first stent 110.

As shown in FIGS. 1A and 1B, the extension 115 has a tubular configuration such that a lumen 115b extends through the extension 115. The lumen 115b is defined by an extension wall 115c. Generally, the extension wall 115c can have any thickness and the thickness may depend on the material used to make the extension wall 115c. In one embodiment, the extension wall 115c has a thickness of about 5 μm to about 50 μm.

The extension 115 may be removably attached to the second end 112 of the first stent 110 by for example mechanical attachment (crimping or swaging), stitching, or fastening by an erodable, degradable, cleavable, or separable fastener or other attachment means. Removable attachments may be facilitated by using a perforated-type attachment or a quick-dissolving solution/film/adhesive. Alternatively, the extension 115 may be more securely attached or affixed to the second end 112 of the first stent 110 by methods such as welding, brazing, fusing, swaging, crimping, stitching, or using fasteners or adhesives. Also, in some embodiments, the second end 112 of the first stent 110 is attached to the extension 115 to allow movement of the first stent 110 relative to the extension 115. In other embodiments, the second end 112 of the first stent 110 is attached to the extension 115 in a manner so that relative movement between the two is not permitted.

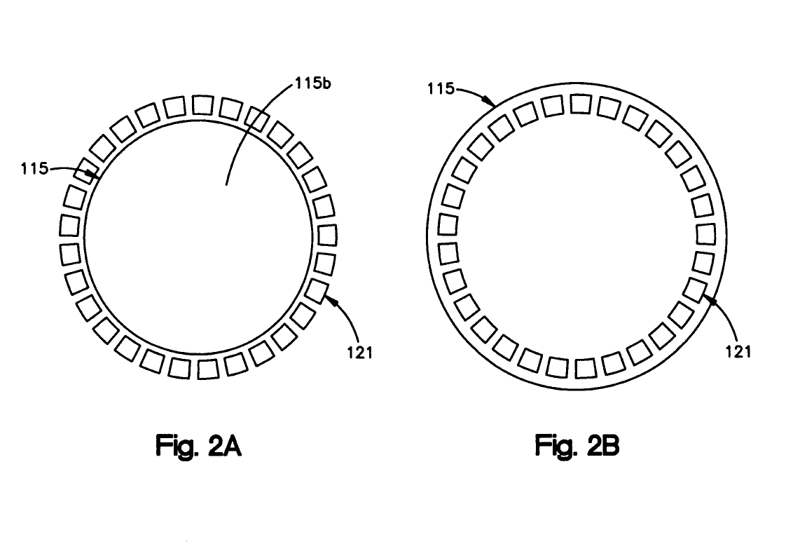

The system 100 also includes a second stent 120 having a surface 123, a first end 121, and a second end 122. As shown in FIG. 1B, the first end 121 of the second stent 120 forms an overlap with at least a portion of the extension 115. As shown in this embodiment, the first end 121 of the second stent 120 forms an overlap with less than the entire extension 115 such that at least a portion of the extension's surface 115a is exposed, i.e., not covered by the second stent. In other embodiments, the first end 121 of the second stent 120 may overlap with the entire extension 115, such that the first and second stents 110 and 120 actually touch each other, or even overlap to some degree. As shown in FIGS. 2A and 2B, the overlap can be formed either with the first end 121 of the second stent 120 overlapping or covering the extension 115, as in FIG. 2A, or with the extension 115 overlapping or covering the first end 121 of the second stent 120, as in FIG. 2B.

The overlap between the extension 115 and the first end 121 of the second stent 120 can be formed prior to delivery of the system 100 to the body lumen. Alternatively, the overlap can be formed when the system 100 is delivered to the lumen. The overlap can be separable or removable such that the extension and the stent end can be readily separated or unconnected. Examples would be a mechanical press-fit or other separable mechanical connection, or connection by means of an erodable or degradable material or fastener. Alternatively, the overlap can be formed by securing the extension 115 to the first end 121 of the second stent 120, by methods such as for example welding, brazing, fusing, swaging, crimping, stitching, or using fasteners or adhesives. A bonding agent such as a polymer could be applied, causing the stent to bond to the extension. The surface tack of the polymer could join the stent and extension upon deployment of the second stent; or a secondary process (UV bond, ultrasound, laser, or other EM energy source) could be used to bond the extension to the stent. In addition, in some embodiments, the first end 121 of the second stent 120 is attached to the extension 115 to allow movement of the second stent 120 relative to the extension 115. In other embodiments, the first end 121 of the second stent 120 is attached to the extension 115 in a manner so that relative movement between the two is not allowed.

The extension can be made from polymers, composites, metals, or a blend of materials (including but not limited to gels, monomers, polymers, composite materials, metals, nano and nano-organic materials such as clays and similar materials, carbon nano tubes, or others). Preferably, the extension is made of a material that provides flexibility to the extension, such as a polymer. Suitable polymers for forming the extension are described below. Also, the material for forming the extension can include a therapeutic agent, examples of which are provided below, so that the therapeutic agent is incorporated into the extension. Alternatively, the therapeutic agent can, instead of or in addition to, be coated onto the extension.

Although FIGS. 1A, 1B, 2A and 2B show the cross-section of extension 115 as being substantially circular in shape, the extension 115 can have other cross-sectional shapes. Other cross-sectional shapes include without limitation, ovals or ellipses, triangles and squares or rectangles. Also, the cross-sectional shape of the extension 115 may be the same as that of the cross-sectional shape of either or both of the first stent 110, or second stent 120. In addition, the cross-sectional shape of the extension 115 may vary along the length of extension 115.

Also, while extension 115 shown in FIGS. 1A and 1B does not have openings in the extension wall 115c, in other embodiments, there may be one or more openings that extend through the extension wall 115c. On the other hand, extension 115 may be provided with openings, for example to adjust its mechanical properties, e.g. flexibility, or to adjust its surface area, or to adjust its drug delivery characteristics (e.g. quantity or area of delivery, or delivery of multiple drugs).

Moreover, even though the stents 110 and 120 shown in FIG. 1A, 1B, 2A and 2B, are indicated as roughly similar in length, the stents used in the system can have different lengths. For example, the first stent 110 can be longer than the second stent 120 or vice versa. Likewise, the diameter of the stents in these figures, which are shown as being similar, can be different. For instance, the diameter of the first stent 110 can be smaller than that of the second stent 120. Also, although the system of FIGS. 1A and 1B show a system comprising two stents, it should be appreciated that in other embodiments, the system can comprise more than two stents.

In some embodiments, such as the one shown in FIGS. 1A and 1B, a coating composition may be disposed on the surface of one or more of the stents. The coating composition can also be disposed on the extension. In the system shown in these figures, a first coating composition 114 is disposed on the surface 113 of the first stent 110 and a second coating composition 124 is disposed on the surface 123 of the second stent 120. In addition, a third coating composition 130 is disposed on the surface 115a of the extension 115. Each of these coating compositions may comprise a polymer and/or a therapeutic agent. Also, some or all of the three coating compositions can be the same, i.e., contain the same amounts of the same constituents. Alternatively, some or all of the three coating compositions can be different, e.g., contain at least one different constituent or contain the same constituents in different amounts.

As shown in FIGS. 1A and 1B, the first and second coating compositions 114, 124 are disposed respectively on the abluminal surfaces of the first and second stents 110, 120. The abluminal surface is the surface of the stent that faces away from the lumen of the stent. In other embodiments, the coating compositions can be disposed on the luminal surface, the surface facing toward the center of the lumen, instead of or in addition to being disposed on the abluminal surface.

Additionally, in certain embodiments, such as that shown in FIGS. 1A and 1B, the stents of the system 100 may have a sidewall structure comprising struts and openings between the struts. In some embodiments, a coating composition is applied to the surface of the stent. When a coating composition is applied to a stent having a sidewall structure with struts and openings, the coating composition may conform to the sidewall structure to preserve the openings, i.e., the openings are not occluded with the coating composition.

In certain embodiments, if the extension is comprised of a polymer, the coating compositions disposed on the surfaces of the stents may comprise the same polymer or a different polymer. Additionally, if the extension comprises a therapeutic agent, the coating compositions disposed on the surfaces of the stents may comprise the same therapeutic agent or a different therapeutic agent. For example, the extension may be formed from a polymer and a therapeutic agent and the coating disposed on the stent comprises the same polymer and the same therapeutic agent.

Moreover, if the same therapeutic agent is incorporated into or coated onto the extension and also used in the coating composition for the stent, the amount or dose of therapeutic agent incorporated into or disposed onto the extension may be the same as or different from the amount or does disposed on the stent. For example, if the stent comprises a sidewall structure having a plurality of struts and openings and in contrast the extension has a continuous surface without openings, it may be desirable to reduce the concentration of the therapeutic agent incorporated into or disposed onto the extension to provide a more uniform delivery of the therapeutic agent from the system. Similarly, if there is a significant overlap between the extension and the stent, it may be desirable to reduce the amount of therapeutic agent incorporated into or disposed onto the portion of the extension that overlaps the stent to avoid delivering too much therapeutic agent from the overlap. Alternatively, it may be desirable to reduce the amount of therapeutic agent disposed onto the portion stent that overlaps the extension.

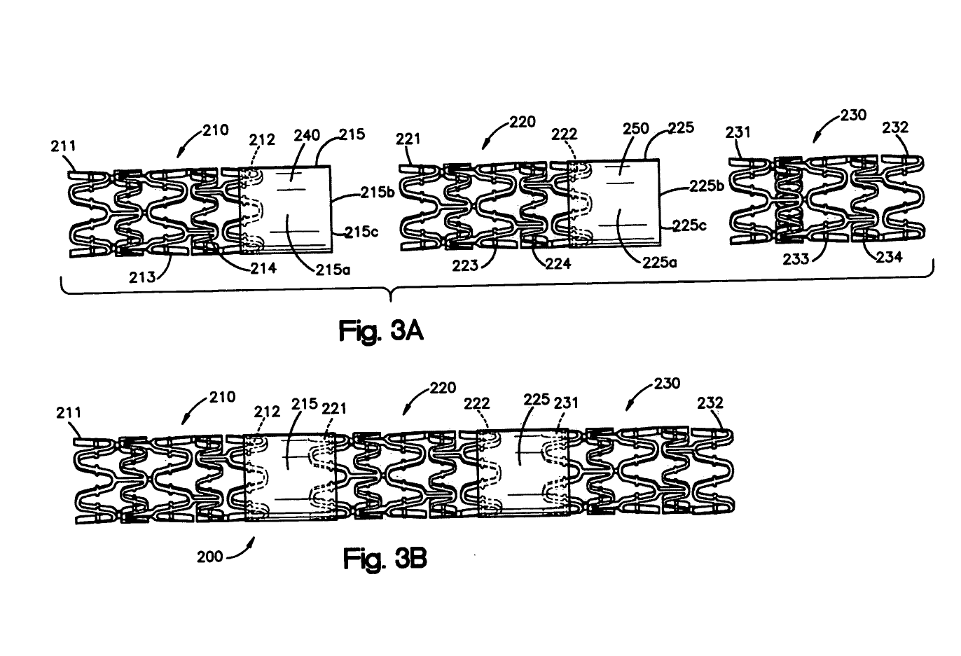

In another embodiment, the system may include a third stent. Such an embodiment is shown in FIGS. 3A and 3B. This system 200 for treating a lumen comprises a first stent 210 with a first extension 215, a second stent 220 with a second extension 225 and a third stent 230. FIG. 3A shows the system 200 when the extensions 215, 225 are not connected to or in contact with respectively the second stent 220 and the third stent 230. FIG. 3B shows the system 200 when the extensions 215, 225 are connected to or in contact respectively with the second stent 220 and third stent 230.

In this system 200, the first stent 210 has a surface 213, a first end 211 and a second end 212. A first extension 215 comprises an extension wall 215c with a surface 215a. The extension wall 215c defines a lumen 215b. The extension 215 is attached or connected to the second end 212 of the first stent 210. The first extension 215 and the second end 212 can be attached according to the methods described above in connection with the embodiment shown in FIGS. 1A-1B and 2A-2B. Furthermore the features and variations discussed with respect to the embodiments shown in FIGS. 1A-1B and 2A-2B can apply to all other embodiments discussed herein, such as that shown in FIGS. 3A and 3B.

The second stent 220 of the system 200 comprises a surface 223, a first end 221 and a second end 222. A second extension 225 has an extension wall 225c with a surface 225a. The extension wall 225c defines a lumen 225b. The extension 225 is attached or connected to the second end 222 of the second stent 220. As shown in FIG. 3B, the first extension 215 and the first end 221 of the second stent 220 form an overlap. As discussed above the overlap can be achieved either with the stent end positioned within the extension, or with the extension positioned within the stent end.

The third stent 230 of the system 200 comprises a surface 233, a first end 231 and a second end 232. As shown in FIG. 3B, the second extension 225 and the first end 231 of the third stent 230 form an overlap.

Also, in the embodiment shown in FIGS. 3A and 3B, coating compositions are disposed on surfaces of one or more of the stents as well as on the extensions. In the system 200, a first coating composition 214 is disposed on the surface 213 of the first stent 210. A second coating composition 224 is disposed on the surface 223 of the second stent 220. A third coating composition 234 is disposed on the surface 233 of the third stent 230. In addition, a fourth and a fifth coating composition 240, 250 are respectively disposed on the surfaces 215a, 225a of the first and second extensions 215, 225. Each of these coating compositions may comprise a polymer and/or a therapeutic agent. Also, some or all of the coating compositions can be the same, i.e., contain the same amounts of the same constituents. Alternatively, some or all of the coating compositions can be different, e.g., contain at least one different constituent or contain the same constituents in different amounts. In other embodiments, some of the stents and/or extensions can be free of a coating composition.

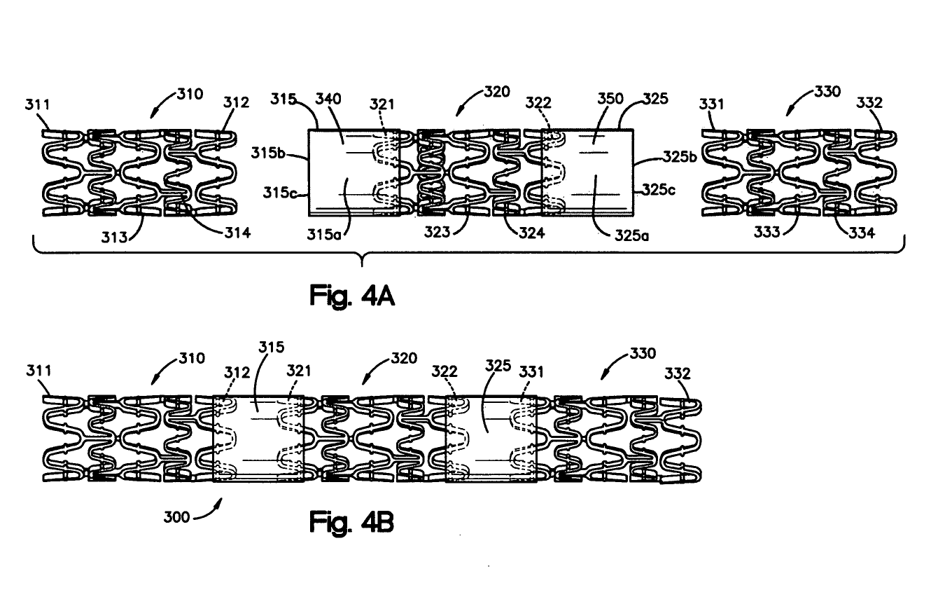

FIGS. 4A and 4B show another embodiment comprising three stents. In this embodiment, the system 300 comprises a first stent 310, a second stent 320 with a first extension 315, a second extension 325 and a third stent 330. FIG. 4A shows the system 300 when the extensions 315, 325 are not connected to or in contact with respectively the first stent 310 and the third stent 330. FIG. 4B shows the system 300 when the extensions 315, 325 are connected to or in contact respectively with the first stent 310 and third stent 330.

In this system 300, the first stent 310 has a surface 313, a first end 311 and a second end 312. The second stent 320 of the system 300 comprises a surface 323, a first end 321 and a second end 322. A first extension 315 comprising an extension wall 315c with a surface 315a is attached to the first end 312 of the second stent 320. The extension wall 315c defines a lumen 315b. A second extension 325 having an extension wall 325c with a surface 325a is attached to the second end 322 of the second stent 320. The extension wall 325c defines a lumen 325b. The third stent 330 of the system 300 comprises a surface 333, a first end 331 and a second end 332.

As shown in FIG. 4B, when the system 300 is in use, the first extension 315 and the second end 312 of the first stent 310 form an overlap. The second extension 325 and the first end 331 of the third stent 330 form an overlap. As discussed above, the overlap can be achieved either with the stent end positioned within the extension, or with the extension positioned within the stent end.

Furthermore, in the system 300, coating compositions are disposed on surfaces of one or more of the stents as well as on one or more of the extensions. In this embodiment, a first coating composition 314 is disposed on the surface 313 of the first stent 310, a second coating composition 324 is disposed on the surface 323 of the second stent 320, and a third coating composition 334 is disposed on the surface 333 of the third stent 330. Also, a fourth and a fifth coating composition 340, 350 are respectively disposed on the surfaces 315a, 325a of the first and second extensions 315, 325. Each of these coating compositions may comprise a polymer and/or a therapeutic agent. Also, some or all of the coating compositions can be the same, i.e., contain the same amounts of the same constituents. Alternatively, some or all of the coating compositions can be different, e.g., contain at least one different constituent or contain the same constituents in different amounts. Also, in some embodiments, some of the stents and/or extensions can be free of a coating composition.

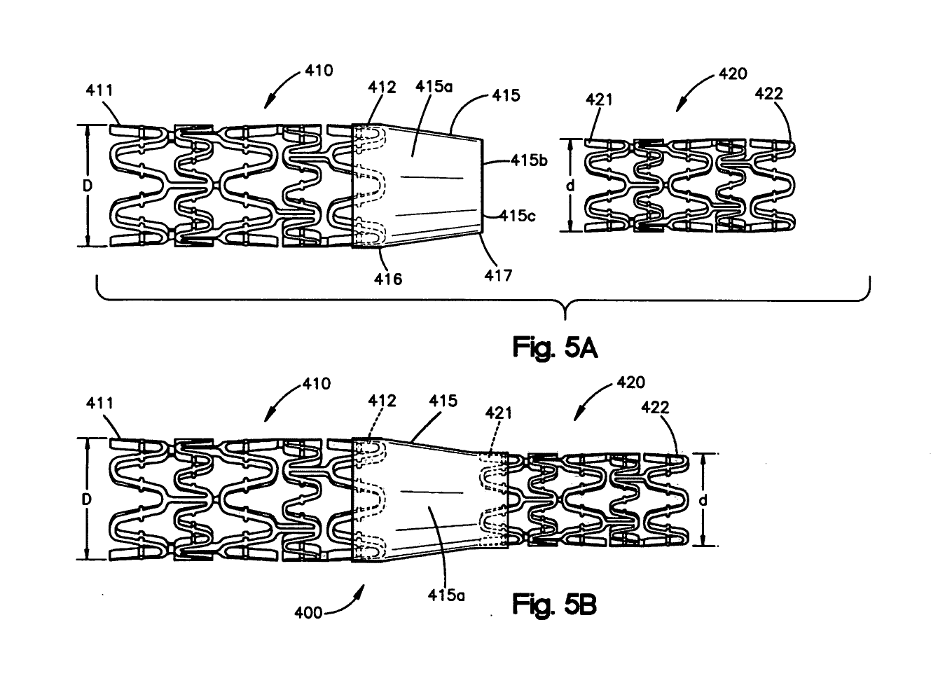

As noted earlier, the stents of the system can have different diameters. In such a situation, the diameter of the extension connecting the stents can vary along the length of the extension. FIGS. 5A and 5B show such an embodiment involving two stents and an extension. In this embodiment, system 400 for treating a lumen comprises a first stent 410 having a diameter D. Although the diameter of this stent is shown as being constant along its length, in other embodiments, the stent diameter can vary along its length. The first stent 410 has a first end 411 and a second end 412. The second end 412 of the first stent 410 is attached to a first extension 415. The first extension 415 comprises an extension wall 415c with a surface 415a. The extension wall 415c defines a lumen 415b. The system 400 also comprises a second stent 420 having a constant diameter d along its length. The second stent 420 comprises a first end 421 and a second end 422.

As shown in FIG. 5B, in this embodiment, the diameter of the portion 416 of the extension 415 that is attached to the second end 412 of the first stent 410 is about the same as the diameter D of the second end 412 of the first stent 410. The diameter of the portion 417 of the extension that forms an overlap with the first end 421 of the second stent 420 is about the same as the diameter d of the first end 421 of the second stent 420. In this embodiment, the second end 412 of the first stent 410 has a larger diameter D than the first end 421 of the second stent 420. When the extension 415 connects these stents, the extension 415 tapers from the second end 412 of the first stent 410 to the first end 421 of the second stent 420. In the example shown, extension 415 has a generally conical shape. However, in other embodiments, the extension may have other shapes, which may depend for example on the cross-sectional shapes of stents 410 and 420.

It should be noted that while the extension 415 has a tapered shape when it forms an overlap with the second stent 420, the extension 415 may not have such a configuration when it does not form the overlap. In particular, FIG. 4A shows the extension 415 as having a tapered shaped even when the extension 415 does not form an overlap with the second stent 420. In alternative embodiments, the extension 415 can have a constant diameter along its length until it forms the overlap. In such an embodiment, the portion of the extension 415 that forms the overlap can be positioned within the second stent 420 so that the extension 415 achieves a tapered shape.

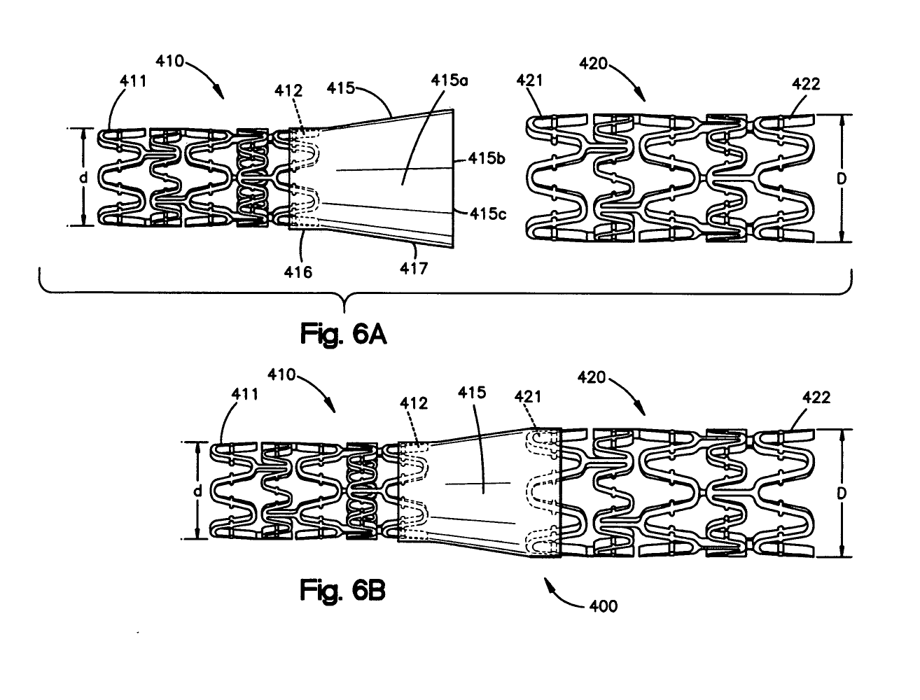

FIGS. 6A and 6B show an embodiment similar to that shown in FIGS. 5A and 5B, however, the second stent has a greater diameter than the first stent. In this embodiment, system 400 for treating a lumen comprises a first stent 410 having a diameter d. Although the diameter of this stent is shown as being constant along its length, in other embodiments, the stent diameter can vary along its length. The first stent 410 has a first end 411 and a second end 412. The second end 412 of the first stent 410 is attached to an extension 415. A first extension 415 comprises an extension wall 415c with a surface 415a. The extension wall 415c defines a lumen 415b. The system 400 also comprises a second stent 420 having a constant diameter D along its length. The second stent 420 comprises a first end 421 and a second end 422.

In this embodiment, the diameter of the portion 416 of the extension that is attached to the second end 412 of the first stent 410 is the same as the diameter d of the second end 412 of the first stent 410 and the diameter of the portion 417 of the extension that forms an overlap with the first end 421 of the second stent 420 is the same as the diameter D of the first end 421 of the second stent 420. In this embodiment, the second end 412 of the first stent 410 has a smaller diameter d than the first end 421 of the second stent 420. When the extension 415 connects these stents, the extension 415 tapers from the first end 421 of the second stent 420 to the second end 412 of the first stent 410

While the extension 415 has a tapered shape when it forms an overlap with the second stent 420, the extension 415 may not have such a configuration when it does not form the overlap. In particular, FIG. 6A shows the extension 415 as having a tapered shaped even when the extension 415 does not form an overlap with the second stent 420. In alternative embodiments, the extension 415 can have a constant diameter along its length until it forms the overlap. In such an embodiment, the portion of the extension 415 that forms the overlap can be stretched so that it can be positioned over the second stent 420, thereby forming a tapered shape.

Although the systems discussed above comprise only 2 or 3 stents, in other embodiments, the system can comprise a greater number of stents connected by extensions. For example, a system may comprise a series of stents of progressively smaller (or larger) diameter connected by extensions. Such a system may be advantageous in treating a body lumen that tapers to a smaller diameter or expands to a larger diameter. In other embodiments, the systems can be useful in treating lumens having a varying diameter along the regions of the lumen that requires treatment.

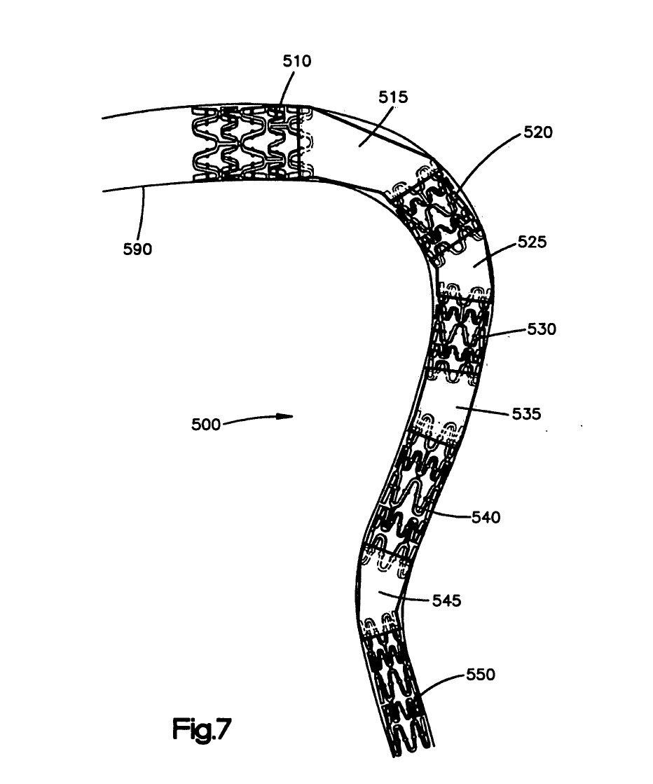

FIG. 7 depicts the use of a system 500 comprising stents and stent extensions to treat a curved and tapered portion of a blood vessel 590. The diameter of the portion of the blood vessel 590 decreases from the top of the portion to the bottom of the portion. System 500 comprises a series of 5 stents 510, 520, 530, 540, 550 of decreasing diameter that are connected to each other by overlaps formed between the ends of the stents and the extensions 515, 525, 535, 545. Extensions 515, 525, 535, 545 are sufficiently flexible to permit system 500 to conform to the curved shape of the blood vessel 590.

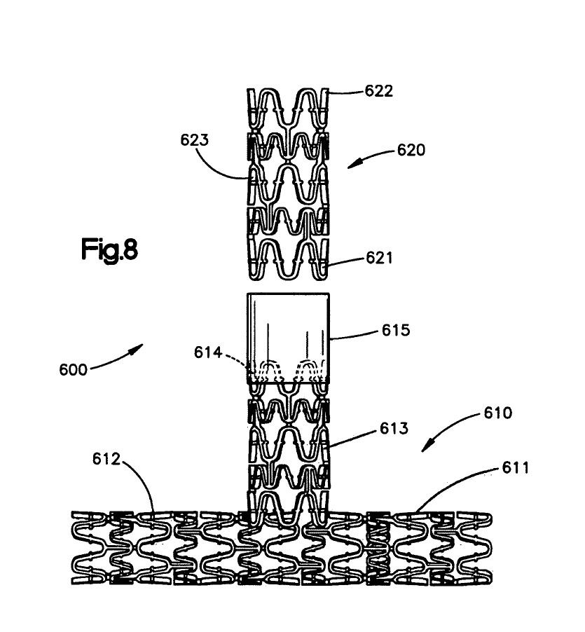

FIG. 8 depicts a system 600 for treating a bifurcated lumen. System 600 comprises a bifurcated stent 610 with an extension 615 and a non-bifurcated stent 620, having ends 621 and 622. Bifurcated stent 610 has a T- or Y-shape. Bifurcated stent 610 comprises tubular portions 611, 612, and 613. In one variation, tubular portions 611, 612, and 613 are all integral with each other. However, in other embodiments, some or all the tubular portions are not integral with one another. For example, all the tubular portions can be connected to each other by connectors. Also, in some embodiments, tubular portions 611 and 612 can be integral with each other while tubular portion 613 is merely connected to one of the other tubular portions, e.g. to tubular portion 611. Extension 615 is attached to end 614 of tubular portion 613, and forms an overlap with an end 621 of the non-bifurcated stent 620. The overlap can be formed with the extension 615 disposed within non-bifurcated stent 620 or alternatively with the end 621 of the non-bifurcated stent 620 within the extension 615.

System 600 can further comprise a graft (not shown). The graft can be disposed in contact with bifurcated stent 610, e.g. covering stent 610 or acting as a lining disposed within stent 610. Alternatively, the graft can be disposed in contact with the non-bifurcated stent 620. Also, the graft can be disposed in contact with more than one component of system 500 or with all components of the system 600.

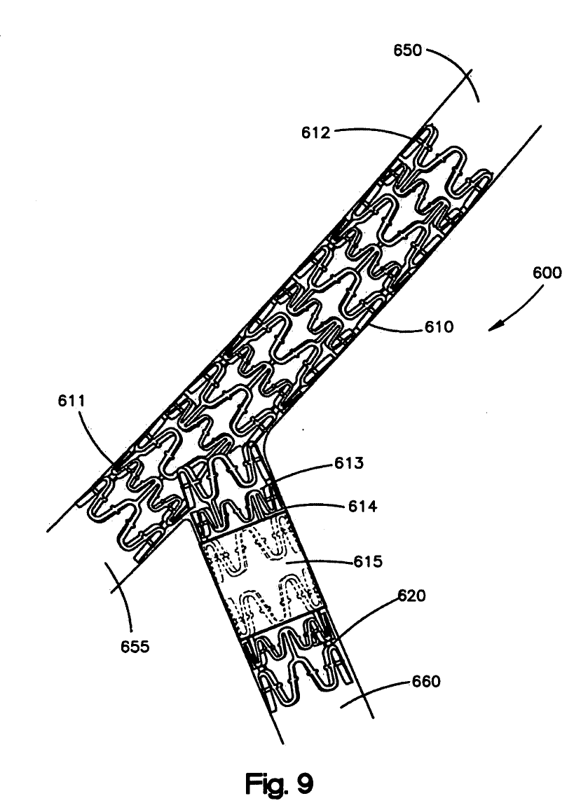

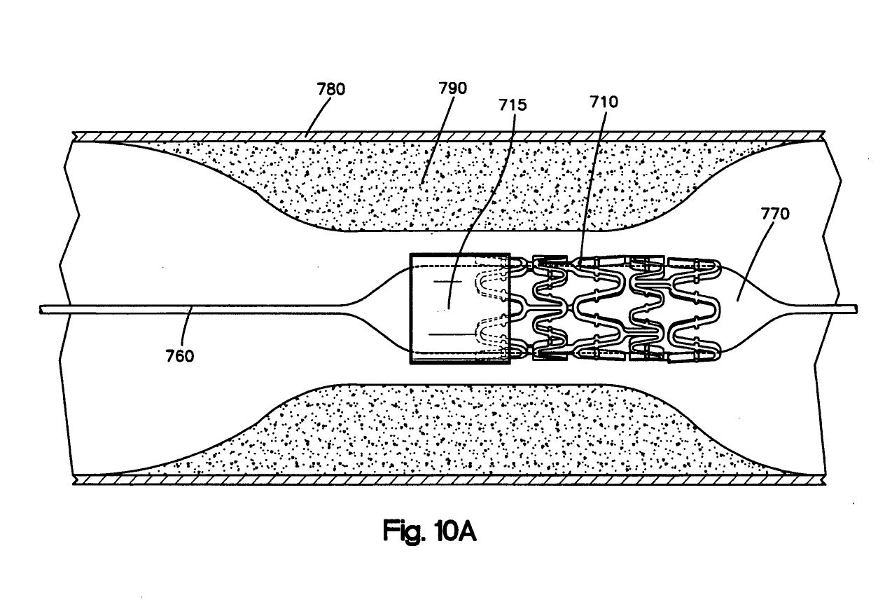

FIG. 9 depicts system 600 of FIG. 8 placed in a bifurcated blood vessel. The bifurcated stent 610 is disposed in the main vessel 650, 655 and side vessel 660. The two stents 610 and 620 are connected to each other by having the extension 615 and the non-bifurcated stent 620 form an overlap. It will be apparent that additional stents and extensions may be used to increase the extent of the system 600 and thereby treat other areas of the main vessel or the side vessels. For example additional extensions can be attached to the ends of tubular portions 611, 612 and such extensions can form overlaps with additional stents.

### A. Stents

Suitable stents for use in the present systems include, for example, vascular stents such as self-expanding stents and balloon expandable stents. Examples of self-expanding stents are illustrated in U.S. Pat. Nos. 4,655,771 and 4,954,126 issued to Wallsten and 5,061,275 issued to Wallsten et al. Examples of appropriate balloon-expandable stents are shown in U.S. Pat. No. 5,449,373 issued to Pinchasik et al. In preferred embodiments, the stent suitable for the present invention is an Express stent. More preferably, the Express stent is an Express™ stent or an Express2™ stent (Boston Scientific, Inc. Natick, Mass.).

The framework of the suitable stents may be formed through various methods as known in the art. The framework may be welded, molded, laser cut, electro-formed, or consist of filaments or fibers which are wound or braided together in order to form a continuous structure.

Stents that are suitable for the present invention may be fabricated from metallic, ceramic, polymeric or composite materials or a combination thereof. Preferably, the materials are biocompatible. Metallic material is more preferable. Suitable metallic materials include metals and alloys based on titanium (such as nitinol, nickel titanium alloys, thermo-memory alloy materials); stainless steel; tantalum, nickel-chrome; or certain cobalt alloys including cobalt-chromium-nickel alloys such as Elgiloy® and Phynox®; PERSS (Platinum EnRiched Stainless Steel) and Niobium. Metallic materials also include clad composite filaments, such as those disclosed in WO 94/16646.

Suitable ceramic materials include, but are not limited to, oxides, carbides, or nitrides of the transition elements such as titanium, hafnium, iridium, chromium, aluminum, and zirconium. Silicon based materials, such as silica, may also be used.

Suitable polymers for forming the stents may be biostable. Also, the polymer may be biodegradable. Suitable polymers include, but are not limited to, styrene isobutylene styrene, polyetheroxides, polyvinyl alcohol, polyglycolic acid, polylactic acid, polyamides, poly-2-hydroxy-butyrate, polycaprolactone, poly(lactic-co-glycolic)acid, and Teflon.

Polymers may be used for forming the stents in the present invention include without limitation isobutylene-based polymers, polystyrene-based polymers, polyacrylates, and polyacrylate derivatives, vinyl acetate-based polymers and its copolymers, polyurethane and its copolymers, silicone and its copolymers, ethylene vinyl-acetate, polyethylene terephtalate, thermoplastic elastomers, polyvinyl chloride, polyolefins, cellulosics, polyamides, polyesters, polysulfones, polytetrafluorethylenes, polycarbonates, acrylonitrile butadiene styrene copolymers, acrylics, polylactic acid, polyglycolic acid, polycaprolactone, polylactic acid-polyethylene oxide copolymers, cellulose, collagens, and chitins.

Other polymers that are useful as materials for making stents include without limitation dacron polyester, poly(ethylene terephthalate), polycarbonate, polymethylmethacrylate, polypropylene, polyalkylene oxalates, polyvinylchloride, polyurethanes, polysiloxanes, nylons, poly(dimethyl siloxane), polycyanoacrylates, polyphosphazenes, poly(amino acids), ethylene glycol I dimethacrylate, poly(methyl methacrylate), poly(2-hydroxyethyl methacrylate), polytetrafluoroethylene poly(HEMA), polyhydroxyalkanoates, polytetrafluorethylene, polycarbonate, poly(glycolide-lactide) co-polymer, polylactic acid, poly(γ-caprolactone), poly(γ-hydroxybutyrate), polydioxanone, poly(γ-ethyl glutamate), polyiminocarbonates, poly(ortho ester), polyanhydrides, alginate, dextran, chitin, cotton, polyglycolic acid, polyurethane, or derivatized versions thereof, i.e., polymers which have been modified to include, for example, attachment sites or cross-linking groups, e.g., RGD, in which the polymers retain their structural integrity while allowing for attachment of cells and molecules, such as proteins, nucleic acids, and the like.

### B. Extensions

The extensions of the present systems can be made of polymers, composites, metals, or a blend of materials (including but not limited to gels, monomers, polymers, composite materials, metals, nano and nano-organic materials such as clays and similar materials, carbon nano tubes, or others). Preferably, the extension is made of a material that provides flexibility to the extension, such as a polymer. Preferably, the extensions are made of co-polymers comprising styrene-isobutyl.

Furthermore, the materials used to make the extensions can include a therapeutic agent such as those listed in Section C below.

The extensions can be formed by spraying, rolling, extruding, casting, injecting, weaving (filaments), drilling or hollowing (in a similar way to making a tube from a rod), or other methods

The extensions can be attached to the stent by applying a bonding agent, such as a polymer, causing the stent to bond to the extension. The surface tack of the polymer could join the stent and extension upon deployment of the second stent; or a secondary process (UV bond, ultrasound, laser, or other EM energy source) could be used to bond the extension to the stent

### C. Therapeutic Agents

The term “therapeutic agent” as used in the present invention encompasses drugs, genetic materials, and biological materials and can be used interchangeably with “biologically active material”. The term “genetic materials” means DNA or RNA, including, without limitation, DNA/RNA encoding a useful protein stated below, intended to be inserted into a human body including viral vectors and non-viral vectors.

The term ‘biological materials’ include cells, yeasts, bacteria, proteins, peptides, cytokines and hormones. Examples for peptides and proteins include vascular endothelial growth factor (VEGF), transforming growth factor (TGF), fibroblast growth factor (FGF), epidermal growth factor (EGF), cartilage growth factor (CGF), nerve growth factor (NGF), keratinocyte growth factor (KGF), skeletal growth factor (SGF), osteoblast-derived growth factor (BDGF), hepatocyte growth factor (HGF), insulin-like growth factor (IGF), cytokine growth factors (CGF), platelet-derived growth factor (PDGF), hypoxia inducible factor-1 (HIF-1), stem cell derived factor (SDF), stem cell factor (SCF), endothelial cell growth supplement (ECGS), granulocyte macrophage colony stimulating factor (GM-CSF), growth differentiation factor (GDF), integrin modulating factor (IMF), calmodulin (CaM), thymidine kinase (TK), tumor necrosis factor (TNF), growth hormone (GH), bone morphogenic protein (BMP) (e.g., BMP-2, BMP-3, BMP-4, BMP-5, BMP-6 (Vgr-1), BMP-7 (PO-1), BMP-8, BMP-9, BMP-10, BMP-11, BMP-12, BMP-14, BMP-15, BMP-16, etc.), matrix metalloproteinase (MMP), tissue inhibitor of matrix metalloproteinase (TIMP), cytokines, interleukin (e.g., IL-1, IL-2, IL-3, IL-4, IL-5, IL-6, IL-7, IL-8, IL-9, IL-10, IL-11, IL-12, IL-15, etc.), lymphokines, interferon, integrin, collagen (all types), elastin, fibrillins, fibronectin, vitronectin, laminin, glycosaminoglycans, proteoglycans, transferrin, cytotactin, cell binding domains (e.g., RGD), and tenascin. Currently preferred BMP's are BMP-2, BMP-3, BMP-4, BMP-5, BMP-6, BMP-7. These dimeric proteins can be provided as homodimers, heterodimers, or combinations thereof, alone or together with other molecules. Cells can be of human origin (autologous or allogeneic) or from an animal source (xenogeneic), genetically engineered, if desired, to deliver proteins of interest at the transplant site. The delivery media can be formulated as needed to maintain cell function and viability. Cells include progenitor cells (e.g., endothelial progenitor cells), stem cells (e.g., mesenchymal, hematopoietic, neuronal), stromal cells, parenchymal cells, undifferentiated cells, fibroblasts, macrophage, and satellite cells.

Other suitable therapeutic agents include:

anti-thrombogenic agents such as heparin, heparin derivatives, urokinase, and PPack (dextrophenylalanine proline arginine chloromethylketone);

anti-proliferative agents such as enoxaprin, angiopeptin, or monoclonal antibodies capable of blocking smooth muscle cell proliferation, hirudin, acetylsalicylic acid, tacrolimus, everolimus, pimecrolimus, sirolimus, zotarolimus, amlodipine and doxazosin;

anti-inflammatory agents such as glucocorticoids, betamethasone, dexamethasone, prednisolone, corticosterone, budesonide, estrogen, sulfasalazine, rosiglitazone, mycophenolic acid and mesalamine;

anti-neoplastic/anti-proliferative/anti-miotic agents such as paclitaxel, 5-fluorouracil, cisplatin, vinblastine, vincristine, epothilones, methotrexate, azathioprine, adriamycin and mutamycin; endostatin, angiostatin and thymidine kinase inhibitors, cladribine, taxol and its analogs or derivatives, paclitaxel as well as its derivatives, analogs or paclitaxel bound to proteins, e.g. Abraxane™;

anesthetic agents such as lidocaine, bupivacaine, and ropivacaine;

anti-coagulants such as D-Phe-Pro-Arg chloromethyl ketone, an RGD peptide-containing compound, heparin, antithrombin compounds, platelet receptor antagonists, anti-thrombin antibodies, anti-platelet receptor antibodies, aspirin (aspirin is also classified as an analgesic, antipyretic and anti-inflammatory drug), dipyridamole, protamine, hirudin, prostaglandin inhibitors, platelet inhibitors, antiplatelet agents such as trapidil or liprostin and tick antiplatelet peptides;

DNA demethylating drugs such as 5-azacytidine, which is also categorized as a RNA or DNA metabolite that inhibit cell growth and induce apoptosis in certain cancer cells;

vascular cell growth promoters such as growth factors, vascular endothelial growth factors (VEGF, all types including VEGF-2), growth factor receptors, transcriptional activators, and translational promoters;

vascular cell growth inhibitors such as anti-proliferative agents, growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin;

cholesterol-lowering agents, vasodilating agents, and agents which interfere with endogenous vasoactive mechanisms;

anti-oxidants, such as probucol;

antibiotic agents, such as penicillin, cefoxitin, oxacillin, tobranycin, rapamycin (sirolimus);

angiogenic substances, such as acidic and basic fibroblast growth factors, estrogen including estradiol (E2), estriol (E3) and 17-beta estradiol;

drugs for heart failure, such as digoxin, beta-blockers, angiotensin-converting enzyme (ACE) inhibitors including captopril and enalopril, statins and related compounds; and

macrolides such as sirolimus or everolimus;

Other therapeutic agents include nitroglycerin, nitrous oxides, nitric oxides, antibiotics, aspirins, digitalis, estrogen, estradiol and glycosides. Preferred therapeutic agents include anti-proliferative drugs such as steroids, vitamins, and restenosis-inhibiting agents. Preferred restenosis-inhibiting agents include microtubule stabilizing agents such as Taxol®, paclitaxel (i.e., paclitaxel, paclitaxel analogs, or paclitaxel derivatives, and mixtures thereof). For example, derivatives suitable for use in the present invention include 2′-succinyl-taxol, 2′-succinyl-taxol triethanolamine, 2′-glutaryl-taxol, 2′-glutaryl-taxol triethanolamine salt, 2′-O-ester with N-(dimethylaminoethyl) glutamine, and 2′-O-ester with N-(dimethylaminoethyl) glutamide hydrochloride salt.

Other preferred therapeutic agents include tacrolimus; halofuginone; inhibitors of HSP90 heat shock proteins such as geldanamycin; microtubule stabilizing agents such as epothilone D; phosphodiesterase inhibitors such as cliostazole; Barkct inhibitors; phospholamban inhibitors; and Serca 2 gene/proteins. In yet another preferred embodiment, the therapeutic agent is an antibiotic such as erythromycin, amphotericin, rapamycin, adriamycin, etc.

In one embodiment, the therapeutic agent is capable of altering the cellular metabolism or inhibiting a cell activity, such as protein synthesis, DNA synthesis, spindle fiber formation, cellular proliferation, cell migration, microtubule formation, microfilament formation, extracellular matrix synthesis, extracellular matrix secretion, or increase in cell volume. In another embodiment, the therapeutic agent is capable of inhibiting cell proliferation and/or migration.

In certain embodiments, the therapeutic agents for use in the medical devices of the present invention can be synthesized by methods well known to one skilled in the art. Alternatively, the therapeutic agents can be purchased from chemical and pharmaceutical companies.

### D. Coating Compositions

The coating compositions of the present invention can comprise a polymer and/or a therapeutic agent, such as those discussed above in Section C. In some embodiments, the therapeutic agent comprises at least 5%, at least 10%, at least 20%, at least 30%, at least 40%, at least 50%, at least 60%, at least 70%, at least 80%, at least 90%, at least 95%, at least 97%, at least 99% or more by weight of the coating composition. Preferably, the therapeutic agent is about 0.01% to about 50% by weight of the coating composition. It is possible, however, to deploy a drug without a carrier polymer, so that the coating is 100% therapeutic agent.

The polymers useful for forming the coating compositions of the present invention should be ones that are biocompatible, particularly during insertion or implantation of the device into the body and avoids irritation to body tissue. Examples of such polymers include, but not limited to, polyurethanes, polyisobutylene and its copolymers, silicones, and polyesters. Other suitable polymers include polyolefins, polyisobutylene, ethylene-alphaolefin copolymers, acrylic polymers and copolymers, vinyl halide polymers and copolymers such as polyvinyl chloride, polyvinyl ethers such as polyvinyl methyl ether, polyvinylidene halides such as polyvinylidene fluoride and polyvinylidene chloride, polyacrylonitrile, polyvinyl ketones, polyvinyl aromatics such as polystyrene, polyvinyl esters such as polyvinyl acetate; copolymers of vinyl monomers, copolymers of vinyl monomers and olefins such as ethylene-methyl methacrylate copolymers, acrylonitrile-styrene copolymers, ABS resins, ethylene-vinyl acetate copolymers, polyamides such as Nylon 66 and polycaprolactone, alkyd resins, polycarbonates, polyoxyethylenes, polyimides, polyethers, epoxy resins, polyurethanes, rayon-triacetate, cellulose, cellulose acetate, cellulose butyrate, cellulose acetate butyrate, cellophane, cellulose nitrate, cellulose propionate, cellulose ethers, carboxymethyl cellulose, collagens, chitins, polylactic acid, polyglycolic acid, and polylactic acid-polyethylene oxide copolymers.

In certain embodiment hydrophobic polymers can be used. Examples of suitable hydrophobic polymers or monomers include, but not limited to, polyolefins, such as polyethylene, polypropylene, poly(1-butene), poly(2-butene), poly(1-pentene), poly(2-pentene), poly(3-methyl-1-pentene), poly(4-methyl-1-pentene), poly(isoprene), poly(4-methyl-1-pentene), ethylene-propylene copolymers, ethylene-propylene-hexadiene copolymers, ethylene-vinyl acetate copolymers, blends of two or more polyolefins and random and block copolymers prepared from two or more different unsaturated monomers; styrene polymers, such as poly(styrene), styrene-isobutylene copolymers, poly(2-methylstyrene), styrene-acrylonitrile copolymers having less than about 20 mole-percent acrylonitrile, and styrene-2,2,3,3,-tetrafluoropropyl methacrylate copolymers; halogenated hydrocarbon polymers, such as poly(chlorotrifluoroethylene), chlorotrifluoroethylene-tetrafluoroethylene copolymers, poly(hexafluoropropylene), poly(tetrafluoroethylene), tetrafluoroethylene, tetrafluoroethylene-ethylene copolymers, poly(trifluoroethylene), poly(vinyl fluoride), and poly(vinylidene fluoride); vinyl polymers, such as poly(vinyl butyrate), poly(vinyl decanoate), poly(vinyl dodecanoate), poly(vinyl hexadecanoate), poly(vinyl hexanoate), poly(vinyl propionate), poly(vinyl octanoate), poly(heptafluoroisopropoxyethylene), poly(heptafluoroisopropoxypropylene), and poly(methacrylonitrile); acrylic polymers, such as poly(n-butyl acetate), poly(ethyl acrylate), poly(1-chlorodifluoromethyl)tetrafluoroethyl acrylate, poly di(chlorofluoromethyl)fluoromethyl acrylate, poly(1,1-dihydroheptafluorobutyl acrylate), poly(1,1-dihydropentafluoroisopropyl acrylate), poly(1,1-dihydropentadecafluorooctyl acrylate), poly(heptafluoroisopropyl acrylate), poly 5-(heptafluoroisopropoxy)pentyl acrylate, poly 11-(heptafluoroisopropoxy)undecyl acrylate, poly 2-(heptafluoropropoxy)ethyl acrylate, and poly(nonafluoroisobutyl acrylate); methacrylic polymers, such as poly(benzyl methacrylate), poly(n-butyl methacrylate), poly(isobutyl methacrylate), poly(t-butyl methacrylate), poly(t-butylaminoethyl methacrylate), poly(dodecyl methacrylate), poly(ethyl methacrylate), poly(2-ethylhexyl methacrylate), poly(n-hexyl methacrylate), poly(phenyl methacry late), poly(n-propyl methacrylate), poly(octadecyl methacrylate), poly(1,1-dihydropentadecafluorooctyl methacrylate), poly(heptafluoroisopropyl methacrylate), poly(heptadecafluorooctyl methacrylate), poly(1-hydrotetrafluoroethyl methacrylate), poly(1,1-dihydrotetrafluoropropyl methacrylate), poly(1-hydrohexafluoroisopropyl methacrylate), and poly(t-nonafluorobutyl methacrylate); polyesters, such a poly(ethylene terephthalate) and poly(butylene terephthalate); condensation type polymers such as and polyurethanes and siloxane-urethane copolymers; polyorganosiloxanes, i.e., polymers characterized by repeating siloxane groups, represented by RaSiO 4-a/2, where R is a monovalent substituted or unsubstituted hydrocarbon radical and the value of a is 1 or 2; and naturally occurring hydrophobic polymers such as rubber.

In alternative embodiments, hydrophilic polymers can be used. Examples of suitable hydrophilic polymers or monomers include, but not limited to; (meth)acrylic acid, or alkaline metal or ammonium salts thereof; (meth)acrylamide; methylenebisacrylamide; (meth)acrylonitrile; polylactic acide; polyglycolic acid; polylactic-glycolic acid; those polymers to which unsaturated dibasic, such as maleic acid and fumaric acid or half esters of these unsaturated dibasic acids, or alkaline metal or ammonium salts of these dibasic adds or half esters, is added; those polymers to which unsaturated sulfonic, such as 2-acrylamido-2-methylpropanesulfonic, 2-(meth)acryloylethanesulfonic acid, or alkaline metal or ammonium salts thereof, is added; and 2-hydroxyethyl (meth)acrylate and 2-hydroxypropyl (meth)acrylate.

Polyvinyl alcohol is also an example of hydrophilic polymer. Polyvinyl alcohol may contain a plurality of hydrophilic groups such as hydroxyl, amido, carboxyl, amino, ammonium or sulfonyl (—SO3). Hydrophilic polymers also include, but are not limited to, starch, polysaccharides and related cellulosic polymers; polyalkylene glycols and oxides such as the polyethylene oxides; polymerized ethylenically unsaturated carboxylic acids such as acrylic, mathacrylic and maleic acids and partial esters derived from these acids and polyhydric alcohols such as the alkylene glycols; homopolymers and copolymers derived from acrylamide; and homopolymers and copolymers of vinylpyrrolidone.

Additional suitable polymers include, but are not limited to, thermoplastic elastomers in general, polyolefins, polyisobutylene, ethylene-alphaolefin copolymers, acrylic polymers and copolymers, vinyl halide polymers and copolymers such as polyvinyl chloride, polyvinyl ethers such as polyvinyl methyl ether, polyvinylidene halides such as polyvinylidene fluoride and polyvinylidene chloride, polyacrylonitrile, polyvinyl ketones, polyvinyl aromatics such as polystyrene, polyvinyl esters such as polyvinyl acetate, copolymers of vinyl monomers, copolymers of vinyl monomers and olefins such as ethylene-methyl methacrylate copolymers, acrylonitrile-styrene copolymers, ABS (acrylonitrile-butadiene-styrene) resins, ethylene-vinyl acetate copolymers, polyamides such as Nylon 66 and polycaprolactone, alkyd resins, polycarbonates, polyoxymethylenes, polyimides, polyethers, polyether block amides, epoxy resins, rayon-triacetate, cellulose, cellulose acetate, cellulose butyrate, cellulose acetate butyrate, cellophane, cellulose nitrate, cellulose propionate, cellulose ethers, carboxymethyl cellulose, collagens, chitins, polylactic acid, polyglycolic acid, polylactic acid-polyethylene oxide copolymers, EPDM (ethylene-propylene-diene) rubbers, fluoropolymers, fluorosilicones, polyethylene glycol, polysaccharides, phospholipids, and combinations of the foregoing. In certain embodiments preferred polymers include, but are not limited to SIBS triblock polymers comprising styrene and isobutylene, or PVDF.

The coating compositions comprising the therapeutic agent and/or polymer can be formed using a solvent. Solvents that may be used to prepare coating compositions include ones which can dissolve or suspend the polymer and/or therapeutic agent in solution. Examples of suitable solvents include, but are not limited to, tetrahydrofuran, methylethylketone, chloroform, toluene, acetone, isooctane, 1,1,1, trichloroethane, dichloromethane, isopropanol, IPA, and mixture thereof.

The coating compositions can be applied to the stents or the extensions by any method. Examples of suitable methods include, but are not limited to, spraying such as by conventional nozzle or ultrasonic nozzle, dipping, rolling, electrostatic deposition, and a batch process such as air suspension, pan coating or ultrasonic mist spraying. Also, more than one coating method can be used.

### E. Delivery of the Stent System

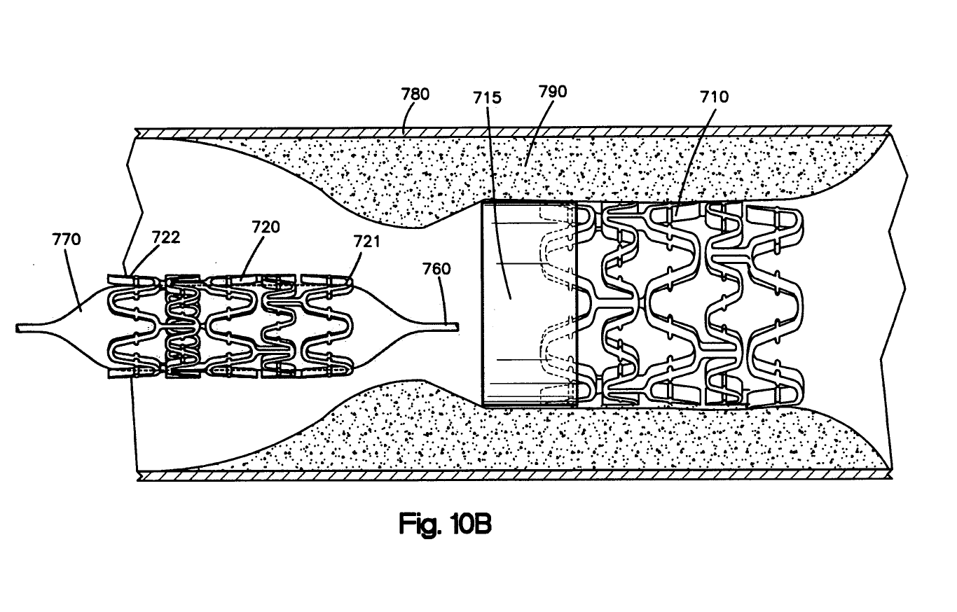

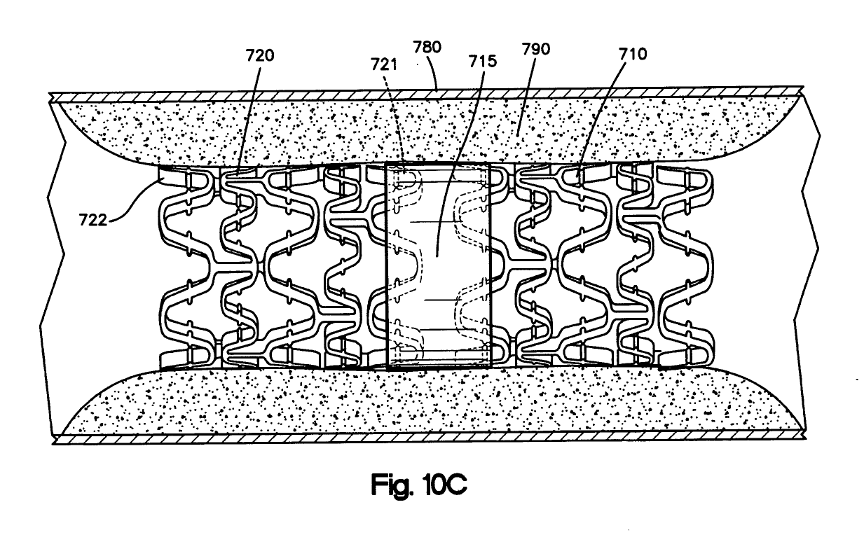

FIGS. 10A-10C show the delivery of a system comprising a first balloon-expandable stent 710 with an extension 715 and a second balloon-expandable stent 720 to a blood vessel 780 with a lesion 790. In FIG. 10A, the first stent 710 is being delivered to the treat the lesion 790 of a blood vessel 780. The first stent 710 is mounted on an inflation balloon 770 of a catheter 760. The balloon 770 is inflated to expand the first stent 710.

FIG. 10B shows the first stent 710 with the extension 715 positioned within the blood vessel 780 at the site of the lesion 790. A second stent 720, having a first end 721 and a second end 722 is mounted on the balloon 770 of the catheter 760 for delivery to the lesion 790. The second end 722 of the second stent 720 will be disposed within the extension 715. FIG. 10C shows both the first and second stents 710, 720 delivered to the site of the lesion 790 in the blood vessel 780. The second end 722 of the second stent 720 and the extension 715 have formed an overlap connecting the first and second stents 710, 720.

The description contained herein is for purposes of illustration and not for purposes of limitation. Changes and modifications may be made to the embodiments of the description and still be within the scope of the invention. Furthermore, obvious changes, modifications or variations will occur to those skilled in the art. Also, all references cited above are incorporated herein, in their entirety, for all purposes related to this disclosure.

What is claimed is:

1. A system for treating a lumen comprising:
a first stent having a surface, a first end, a second end, and a coating composition comprising a first polymer and a first therapeutic agent disposed on the surface;
a first tubular extension attached to the second end of the first stent, wherein the first extension comprises a second polymer and a second therapeutic agent and
a second stent having a surface, a first end and a second end, wherein the first end of the second stent forms an overlap with the first extension.

2. The system of claim 1, wherein the first extension is disposed within the first end of the second stent.

3. The system of claim 1, wherein the first end of the second stent is disposed within the first extension.

4. The system of claim 1, wherein the first end of the second stent forms an overlap with less than the entire first extension.

5. The system of claim 1 wherein the first and second polymers are the same.

6. The system of claim 1, wherein the first and second therapeutic agents are the same.

7. The system of claim 1 wherein the surface of the first stent is an abluminal surface.

8. The system of claim 1 wherein the second stent further comprises the coating composition disposed on the surface of the second stent.

9. The system of claim 8 wherein the surface of the second stent is an abluminal surface.

10. The system of claim 1 wherein the first and second stents are intravascular stents.

11. The system of claim 1, wherein the first stent comprises a stent sidewall structure comprising a plurality of struts and a plurality of openings therein; and wherein the coating composition conforms to the surface to preserve the openings.

12. The system of claim 1, wherein the first stent comprises a metal.

13. The system of claim 1, wherein the first extension has a thickness of about 5 μm to about 50 μm.

14. The system of claim 1 wherein the second stent further comprises a second tubular extension attached to the second end of the second stent, wherein the second extension comprises a third polymer and a third therapeutic agent.

15. The system of claim 14, wherein the third polymer is the same as the second polymer.

16. The system of claim 14, wherein the third therapeutic agent is the same as the second therapeutic agent.

17. The system of claim 14, further comprising a third stent having a first end and a second end, wherein the third stent forms an overlap with the second extension.

18. The system of claim 17, wherein the second extension is disposed within the first end of the third stent.

19. The system of claim 17, wherein the first end of the third stent is disposed within the second extension.

20. The system of claim 17, wherein the third stent forms an overlap with less than the entire second extension.

21. The system of claim 1 wherein the first stent further comprises a second tubular extension attached to the first end of the first stent, wherein the second extension comprises a third polymer and a third therapeutic agent.

22. The system of claim 21, wherein the third polymer is the same as the second polymer.

23. The system of claim 21, wherein the third therapeutic agent is the same as the second therapeutic agent.

24. The system of claim 21, further comprising a third stent having a first end and a second end, wherein the second end of the third stent forms an overlap with the second extension.

25. The system of claim 24, wherein the second extension is disposed within the second end of the third stent.

26. The system of claim 24, wherein the second end of the third stent is disposed within the second extension.

27. The system of claim 24, wherein the second end of the third stent forms an overlap with less than the entire second extension.

28. The system of claim 1, wherein the first therapeutic agent comprises paclitaxel or everolimus.

29. The system of claim 1, wherein the first polymer comprises PVDF.

30. The system of claim 1, wherein the second end of the first stent has a first diameter and the first end of the second stent has a second diameter, and wherein the first diameter is different from the second diameter.

31. The system of claim 30, wherein the first diameter is greater than the second diameter.

32. The system of claim 1, wherein the first extension comprises a first end having a first diameter and a second end comprising a second diameter; wherein the first end of the first extension is attached to the second end of the first stent; and wherein the first diameter is different from the second diameter prior to the first extension being attached to the second end of the first stent.

33. A system for treating a blood vessel comprising:
a first intravascular metal stent having a surface, a first end, a second end, and a coating composition comprising a first polymer and an agent for inhibiting the proliferation of smooth muscle cells disposed on the surface; and
a first tubular extension attached to the second end of the first stent, wherein the first extension comprises a second polymer and the agent for inhibiting the proliferation of smooth muscle cells; and
a second intravascular metal stent having a surface, the coating composition disposed on the surface of the second stent, a first end and a second end, wherein the first end of the second stent forms an overlap with less than the entire first extension.

34. A system for treating a blood vessel comprising:
a first intravascular metal stent having an abluminal surface, a first end, a second end, and a coating composition comprising a first biostable polymer and an anti-restenosis agent disposed on the abluminal surface; and
a first tubular extension attached to the second end, wherein the first extension comprises a second biostable polymer and the anti-restenosis agent and
a second intravascular metal stent having an abluminal surface, a first end and a second end, wherein the first end of the second stent forms an overlap with less than the entire first extension.

35. A system for treating a bifurcated lumen comprising:
a bifurcated stent comprising a surface, a first tubular portion, a second tubular portion, a third tubular portion having an end and a tubular extension attached to the end of the third tubular portion; and
a non-bifurcated stent comprising a surface, a first end and a second end, wherein the first end of the non-bifurcated stent forms an overlap the extension.

36. The system of claim 35, wherein the extension is disposed within the first end of the non-bifurcated stent.

37. The system of claim 35, wherein the first end of the non-bifurcated stent is disposed within the extension.

38. The system of claim 35, wherein the first end of the non-bifurcated stent forms an overlap with less than the entire extension.

39. The system of claim 35, wherein the extension comprises a first polymer

40. The system of claim 35, wherein the extension comprises a first therapeutic agent.

41. The system of claim 35 further comprising a graft disposed in contact with the bifurcated stent.

42. The system of claim 41 wherein the graft comprises a first polymer.

43. The system of claim 42 wherein the bifurcated stent further comprises a coating composition disposed on the surface of the bifurcated stent.

44. The system of claim 43 wherein the coating composition comprises a second polymer.

45. The system of claim 44 wherein the first and second polymers are the same.

46. The system of claim 43 wherein the coating composition comprises a therapeutic agent.

47. The system of claim 46 wherein the therapeutic agents comprise an anti-thrombogenic agent, anti-angiogenesis agent, anti-proliferative agent, antibiotic, anti-restenosis agent, growth factor, immunosuppressant, or radiochemical.

48. The system of claim 43 wherein the surface of the bifurcated stent is an abluminal surface.

49. The system of claim 35 wherein the non-bifurcated stent further comprises the coating composition disposed on the surface of the non-bifurcated stent.

50. The system of claim 43, wherein the bifurcated stent and the non-bifurcated stent each comprises a metal.

51. The system of claim 35, wherein the extension has a thickness of about 5 μm to about 50 μm.

52. The system of claim 35 wherein the first tubular portion, the second tubular portion and the third tubular portion are all integral with each other.

53. The system of claim 35 wherein the first tubular portion and the second tubular portion are integral with each other and the third tubular portion is connected to the first tubular portion.

54. A system for treating a bifurcated blood vessel comprising:
a bifurcated intravascular metal stent comprising a surface, a first tubular portion, a second tubular portion, a third tubular portion having an end and a tubular extension attached to the end of the third tubular portion; wherein the extension comprises a first polymer and
a non-bifurcated intravascular metal stent comprising a surface, a first end and a second end, wherein the first end of the non-bifurcated stent forms an overlap with less than the entire extension; and
a graft comprising the first polymer disposed within the bifurcated stent.
